(19) United States
(12) Patent Application Publication
(10) Pub. No.: US 2008/0213174 A1
(43) Pub. Date: Sep. 4, 2008

(54) Radiolabelled Insulin

(76) Inventors: Matthias Eberhard Glaser, London (GB); Frank Brady, London (GB); Sajinder Kaur Luthra, London (GB); Alan Cuthbertson, Oslo (NO); Hege Karlsen, Oslo (NO)

Correspondence Address: GE HEALTHCARE, INC., IP DEPARTMENT, 101 CARNEGIE CENTER, PRINCETON, NJ, 08540-6231, US

(21) Appl. No.: 11/575,158

(22) PCT Filed: Sep. 13, 2005
(86) PCT No.: PCT/GB2005/003527

§ 371 Date: Dec. 12, 2007

(30) Foreign Application Priority Data
Sep. 14, 2004 (GB) 0420365.9

(51) Int. Cl. A61K 51/08; C07K 14/62

(52) U.S. Cl. 424/1.69; 530/303

(57) ABSTRACT

The invention relates to in vivo imaging agents, specifically radiolabelled insulin derivatives of formula (III); wherein X is —CO—NH—, —NH—, —O—, —NHCONH—, or —NHCSNH—, and is preferably —CO—NH—, —NH— or —O—; Y is H, alkyl or aryl substituents; R* is a radiolabel moiety suitable for detection by SPECT or PET; and methods for preparing the same as well as their use in in vivo imaging methods.

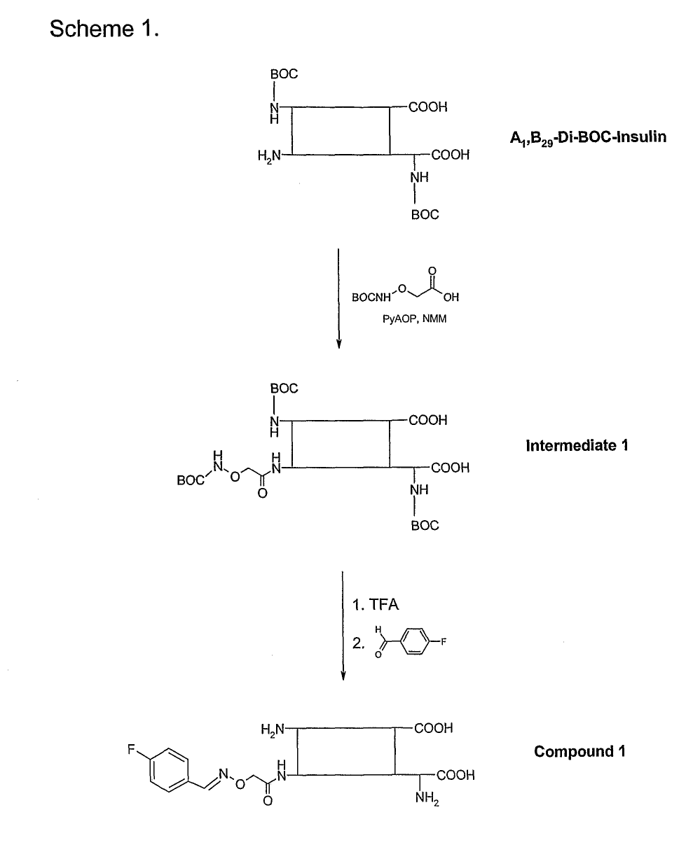

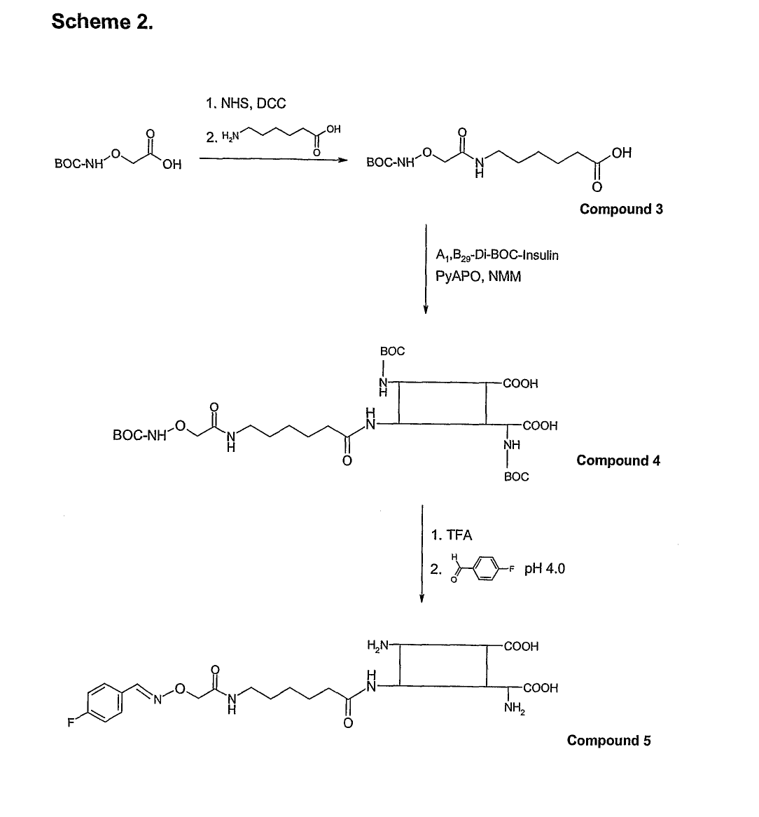

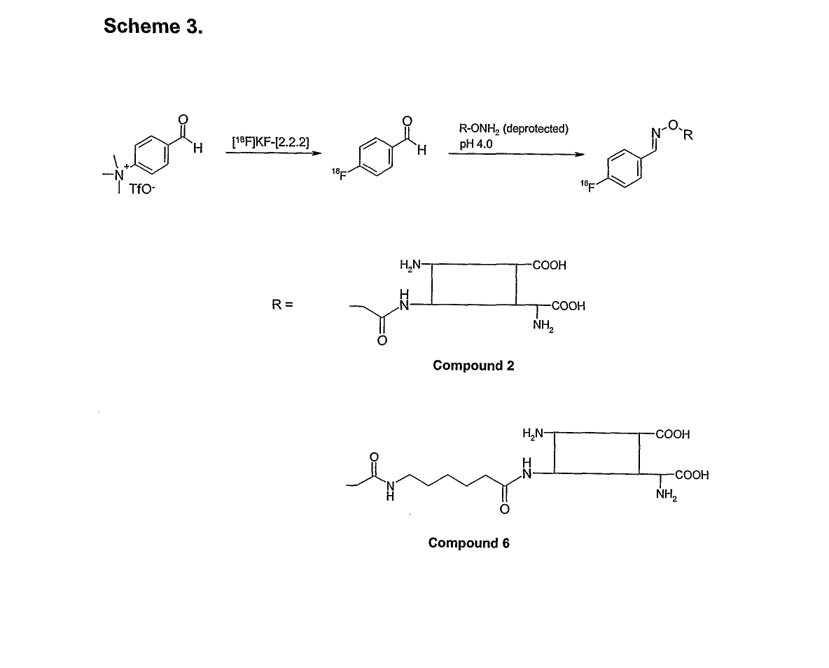

The present invention relates to in vivo imaging agents, specifically radiolabelled insulin and methods for preparing the same. Radiolabelled insulin has use for diagnostic imaging using single-photon emission tomography (SPECT) or positron emission tomography (PET) as well as for studying insulin receptors and their ligand interactions in vivo.

Insulin is a polypeptide hormone produced by the pancreatic beta cells. Insulin regulates carbohydrate and lipid metabolism and influences protein synthesis. Recent literature (New Scientist, Aug. 14, 2004, p34) further suggests that cellular energy levels may influence cell growth and proliferation such that an imaging agent selective for the insulin receptor may have utility in imaging and diagnosis of cancer. Insulin is an active dimer composed of 51 amino acid residues. Natural insulin (from human, bovine, or porcine source), recombinant insulin, and semi-synthetic human insulin are commonly used in the management of diabetes. Radiolabelled insulin, for example, incorporating a radioiodine label is known in the art and has been used, for example, to study insulin metabolism and in in vivo receptor binding experiments. The application of radiolabelled bioactive peptides for diagnostic and research imaging is gaining importance in the field of nuclear medicine. ¹⁸F, with its half-life of approximately 110 minutes, is the positron-emitting nuclide of choice for many imaging studies and diagnostic procedures. ¹⁸F needs to be incorporated into bioactive peptides rapidly, efficiently, and in such a way that the ¹⁸F-labelled product retains biological activity. One report of an ¹⁸F-labelled insulin (Shai et al, Biochemistry (1989), 28, 4801-6) uses a 4-(fluoromethyl)benzoyl synthon to label the B, position of human insulin. However, the radiosynthesis is complicated and time-consuming. Therefore, there exists a need for further methods of radiolabelling (including ¹⁸F-labelling) insulin and for novel radiolabelled (including ¹⁸F-labelled) insulin imaging agents.

Thus, according to one aspect of the invention, there is provided a method for radiolabelling comprising reaction of a compound of formula (I) with a compound of formula (II)

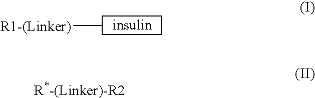

wherein

R1 is a functional group which reacts site-specifically with R2. R1 can be ammonia derivatives such as primary amine, secondary amine, hydroxylamine, hydrazine, hydrazide, aminoxy, phenylhydrazine, semicarbazide, or thiosemicarbazide, and is preferably a hydrazine, hydrazide or aminoxy group;

R2 is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;

R* is a radiolabel moiety suitable for detection by SPECT or PET;

to give a conjugate of formula (III):

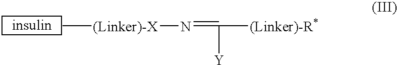

wherein X is —CO—NH—, —NH—, —O—, —NHCONH—, or —NHCSNH—, and is preferably —CO—NH—, —NH— or —O—; Y is H, alkyl or aryl substituents; R* is as defined for the compound of formula (II).

The reaction may be effected in a suitable solvent, for example, in an aqueous buffer in the pH range 2 to 11, suitably 3 to 11, more suitably 3 to 6, and at a non-extreme temperature of from 5 to 80° C., preferably at ambient temperature.

The Linker group in the compounds of formulae (I) and (II) are each independently a C₁₋₆₀ hydrocarbyl group, suitably a C₁₋₃₀ hydrocarbyl group, optionally including 1 to 30 heteroatoms, suitably 1 to 10 heteroatoms such as oxygen or nitrogen.

Suitable Linker groups include alkyl, alkenyl, alkynyl chains, aromatic, polyaromatic, and heteroaromatic rings, and polymers comprising ethyleneglycol, amino acid, or carbohydrate subunits.

The term “hydrocarbyl group” means an organic substituent consisting of carbon and hydrogen, such groups may include saturated, unsaturated, or aromatic portions.

R1 in the compounds of formula (I) and related aspects of the invention is preferably selected from —NHNH₂, —C(O)NHNH₂, and —ONH₂ and is preferably —ONH₂.

R2 in the compounds of formula (II) and related aspects of the invention are each preferably selected from —CHO, >C=O, —CH(—O—C₁₋₄alkyl—O—) such as —CH(—OCH₂CH₂O—), and —CH(OC₁₋₄alkyl)₂ such as —CH(OCH₃)₂, and in a preferred aspect R2 is —CHO.

Suitably, the R2 aldehyde is generated by in situ oxidation of a precursor functionalised vector containing a 1,2-diol or 1,2 aminoalcohol group. For example, the latter can be inserted into the peptide sequence directly during synthesis using the amino acid Fmoc-Dpr(Boc-Ser)-OH described by Wahl et al in Tetrahedron Letts. 37, 6861 (1996). Suitable oxidising agents which may be used to generate the R2 moiety in the compound of formula (II), include periodate, periodic acid, paraperiodic acid, sodium metaperiodate, and potassium metaperiodate.

R* is a radiolabel moiety suitable for detection by SPECT or PET, preferably R* is ¹⁸F, radioiodine (¹²³I, ¹²⁴I, ¹²⁵I, or ¹³¹I), ⁷⁵Br, or a ¹¹C containing group such as [¹¹C]C₁₋₆alkylamine, most preferably, R* is ¹⁸F.

Y in the compound of formula (III) and related aspects of the invention is preferably H, C₁₋₆alkyl (such as methyl), or phenyl.

In one aspect of the invention, the compound of formula (II) is of formula (IIa):

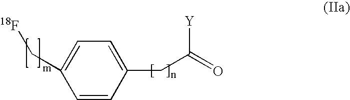

wherein m is an integer of 0 to 10, n is an integer of from 0 to 20, and Y is hydrogen, C₁₋₆alkyl (such as methyl), or phenyl. Preferably, in the compound of formula (IIa), m is 0, n is 0, and Y is hydrogen such that the compound of formula (IIa) is 4-[¹⁸F]fluorobenzaldehyde.

In one aspect of the invention, the compound of formula (I) is of formula (Ia):

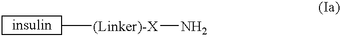

wherein X is —CO—NH—, —NH— or —0— and is preferably —O—.

Preferred linkers in the compounds of formula (I) and (Ia) include: —C(O)—(C₁₋₂₀alkyl)—NHC(O)CH₂- and —C(O)—(C₁₋₂₀alkyl)-.

Thus, in a preferred aspect, the present invention provides a method for radiofluorination comprising reaction of a compound of formula (Ia):

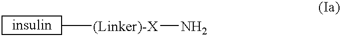

wherein X is —CO—NH—, —NH— or —O— and is preferably —O—; with a compound of formula (IIa):

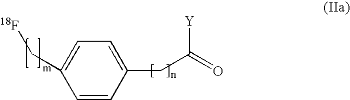

wherein m is an integer of 0 to 10 (preferably 0), n is an integer of from 0 to 20 (preferably 0), and Y is hydrogen, C₁₋₆alkyl (such as methyl), or phenyl (Y is preferably hydrogen);

to give a compound of formula (IIIa):

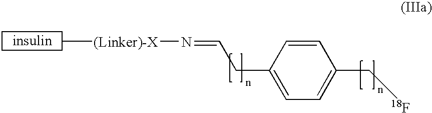

wherein X is as defined for the compound of formula (Ia), m and n are defined as for the compound of formula (IIa).

In a further preferred aspect, the present invention provides a method for radiofluorination comprising reaction of a compound of formula (Ib):

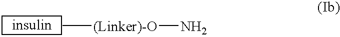

with a compound of formula (IIb):

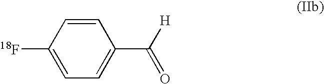

to give a compound of formula (IIIb):

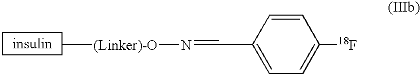

The Linker groups in the compounds of formulae (I), (Ia), (Ib), (II), (IIa), and (IIb) are chosen to maximise efficiency of the radiofluorination reaction and to provide good in vivo pharmacokinetics, such as favourable excretion characteristics in the resultant conjugate of formula (III), (IIIa), or (IIIb). The use of linker groups with different lipophilicities and or charge can significantly change the in vivo pharmacokinetics of the peptide to suit the imaging need. For example, where it is desirable for a conjugate of formula (III), (IIIa), (IIIb) to be cleared from the body by renal excretion, a hydrophilic linker is used, and where it is desirable for clearance to be by hepatobiliary excretion a hydrophobic linker is used. Linkers including a polyethylene glycol moiety have been found to slow blood clearance which is desirable in some circumstances.

In the compounds of formulae (I), (Ia),(Ib), (III), (IIIa), and (IIIb), the insulin may be human, bovine, porcine though is suitably human insulin; and may be natural, recombinant, or synthetic. The insulin may alternatively be an analogue of natural insulin, such as those described in U.S. Pat. No. 5,656,722, DE 3,837,825, WO 95/07931, EP 383472, or J Brange et al, Nature 333 (1988), 679.

The compounds of formula (I), (Ia), and (Ib) may be prepared by standard methods of peptide synthesis, for example, solid-phase peptide synthesis, for example, as described in Atherton, E. and Sheppard, R. C.; “Solid Phase Synthesis”; IRL Press: Oxford, 1989. Incorporation of the group R1 in a compound of formula (I), (Ia), or (Ib) may be achieved by reaction of a free primary amino group of the peptide, modification of which does not affect the binding characteristics of the insulin. The functional groups R1 is preferably introduced by formation of a stable amide bond formed by reaction of a peptide amine function with an activated carboxylic acid and introduced either during or following the peptide synthesis. As would be apparent to a person skilled in the art, during introduction of R1 to a compound of formula (I), (Ia), or (Ib) certain functional groups in the insulin may need to be protected. Suitable protection and deprotection methodologies may be found, for example, in “Protecting Groups in Organic Synthesis”, Theodora W. Greene and Peter G. M. Wuts, published by John Wiley & Sons Inc. One particularly useful protected insulin intermediate which may be modified by addition of a Linker and R1 group, is A₁, B₂₉-diBoc-insulin which may be prepared by the methods described in Shai et al, Biochemistry (1989),28,4801-6. The Boc (tert-butoxycarbonyl) protecting groups may be removed prior to the radiofluorination reaction by hydrolysis, for example with an acid such as trifluoroacetic acid.

In another aspect, the present invention provides a compound of formulae (I), (Ia) or (Ib) as defined above and protected derivatives thereof. Preferred compounds of formula (I), (Ia), and (Ib) are those in which the insulin is human insulin. Compounds of formula (I), (Ia), and (Ib) have use as precursors for synthesis of PET imaging agents and diagnostics and may, for example, be provided in kit form ready for radiofluorination according to the above methods.

Compounds of formula (II), (IIa), and (IIb) in which R* is ¹⁸F, may be prepared as described in international patent application WO 2004/080492. Compounds of formula (II) in which R* is radioiodine may be prepared from the corresponding trialkyl tin precursor by reaction with a radioiodide salt, suitably an alkali metal iodide such as sodium iodide in the presence of an acid such as peracetic acid. Compounds of formula (II) in which R* is a ¹¹C-containing group such as a ¹¹C-alkylamine group, may be prepared, for example, by ¹¹C-alkylation of a corresponding primary amine. A thorough review of such ¹¹C-labelling techniques may be found in Antoni et al “Aspects on the Synthesis of ¹¹C-Labelled Compounds” in Handbook of Radiopharmaceuticals, Ed. M. J. Welch and C. S. Redvanly (2003, John Wiley and Sons).

In a further aspect, the present invention provides radiolabelled conjugates of formula (III), (IIIa), or (IIIb) as defined above. Preferred compounds of formula 15 (III), (IIa), or (IIIb) are those in which the insulin is human insulin.

The present invention also provides a radiopharmaceutical composition comprising an effective amount (e.g. an amount effective for use in in vivo PET imaging) of a compound of formula (III), (IIIa), or (IIIb) as defined above, together with one or more pharmaceutically acceptable adjuvants, excipients, or diluents.

A preferred embodiment of the invention relates to a compound of general formula (III), (IIIa), or (IIIb) as defined above, for medical use and particularly for use in in vivo imaging by SPECT or PET, suitably for in vivo imaging or diagnosis of a disease in which insulin is implicated, for example, myocardial insulin resistance, cardiac hypertrophy, hypertension, cancer, and type II diabetes.

The radiolabelled conjugates of formula (III), (IIIa), or (IIIb) may be administered to patients for SPECT or PET imaging in amounts sufficient to yield the desired signal, typical radionuclide dosages of 0.01 to 100 mCi, preferably 0.1 to 50 mCi will normally be sufficient per 70 kg bodyweight.

The radiolabelled conjugates of formula (III), (IIIa), or (IIIb) may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

Viewed from a further aspect the invention provides the use of a radiolabelled conjugate of formula (III), (IIIa), or (IIIb) for the manufacture of a radiopharmaceutical for use in a method of in vivo imaging, suitably SPECT or PET, and preferably for imaging a disease in which insulin is implicated; involving administration of said radiopharmaceutical to a human or animal body and generation of an image of at least part of said body.

Viewed from a still further aspect the invention provides a method of generating an image of a human or animal body involving administering a radiopharmaceutical to said body, e.g. into the vascular system and generating an image of at least a part of said body to which said radiopharmaceutical has distributed using SPECT or PET, wherein said radiopharmaceutical comprises a radiolabelled conjugate of formula (III), (IIIa), or (IIIb).

Viewed from a further aspect the invention provides a method of monitoring the effect of treatment of a human or animal body with a drug to combat a condition associated with insulin, said method comprising administering to said body a radiolabelled conjugate of formula (III), (IIIa), or (IIIb) and detecting the uptake of said conjugate, said administration and detection optionally but preferably being effected repeatedly, e.g. before, during and after treatment with said drug.

In yet another embodiment of the instant invention, there is provided a kit for the preparation of a radiofluorinated tracer comprising a prosthetic group of formula (II), (IIa), or (IIb) and a compound of formula (I), (Ia), or (Ib).

### EXAMPLES

The invention is illustrated by way of examples in which the following abbreviations are used:
HPLC: high performance liquid chromatography
TFA: trifluoroacetic acid
UV: ultraviolet
DMF: N, N-dimethylformamide
BOC: t-butoxycarbonyl
DMSO: dimethyl sulphoxide
   Preparation of ¹⁸F-B₁-modified insulin using aminooxy conjugation

### EXPERIMENTAL

### Materials

A₁,B₂₉-di-BOC-insulin is prepared using human recombinant insulin (Sigma) according to the method by Shai et al. [Biochemistry 28 (1989) 4801]. BOG-aminooxyacetic acid is obtained from Fluka. (4-Aza-1,2,3-benzotriazol-3-yloxy)-tris(pyrrolidino) phosphonium hexafluorophosphate (PyAOP), 4-fluorobenzaldehyde, Kryptofix®, and N-methylmorpholine (NMM), anhydrous acetonitrile, and anhydrous dimethylsulfoxide are obtained from Sigma-Aldrich.

Preparation of B₁-BOC-aminooxy-acetyl-A₁, B₂₉-di-BOC-insulin (Intermediate 1, Scheme 1)

### Preparative HPLC

Column: Phenomenex Luna prep. C18; A: water (0.1% TFA), B: MeCN (0.1% TFA),

Flow rate: 5 ml/min, gradient: 30-60% B in 30 min, UV detector: 214 nm

### Analytical HPLC

Column: Phenomenex Luna C18; A: water (0.1% TFA), B: MeCN (0.1% TFA),

Flow rate: 1 ml/min, gradient: 25-80% B in 20 min, UV detector: 214 and 254 nm

### Preparation

A₁,B₂₉-di-BOC-insulin (10 mg, 1.7 μmol) in DMF is added to a solution of BOC-aminooxyacetic acid (1.3 mg, 6.6 μmol), PyAOP (3.4 mg, 6.6 μmol) and NMM (1.3 mg, 1.5 μl, 13 μmol) in DMF (1.5 ml). After 4 hours the DMF is evaporated under reduced pressure and the crude product purified using preparative HPLC (yield: 5.3 mg, 51%).

### Preparation of B₁-(4-Fluoro-Benzylideneaminooxv)-Acetyl-Insulin (Compound 1, Scheme 1)

To the BOC-protected insulin derivative Intermediate 1 (5 mg) is added a solution of TFA/5% water (0.2 ml). After standing one minute at room temperature, the liquid phase is evaporated by a stream of nitrogen. The residue is taken up in ammonium acetate buffer (pH 4.0, 0.5 mM) and reacted with one equivalent of 4-fluorobenzaldehyde. The product is purified by preparative HPLC and characterised by LC-MS.

### Preparation of B₁-(4-[¹⁸F]Fluoro-Benzylideneaminooxy)-Acetyl-Insulin (Compound 2, Scheme 3)

### [¹⁸F]Fluorobenzaldehyde

[¹⁸F]Fluorobenzaldehyde is prepared following the method by S. M. Haka et a.l [J. Labelled Cpd. and Radiopharm. 27 (1989) 823]. Briefly, ¹⁸F-fluorine is obtained from a cyclotron using the ¹⁸O(p,n)¹⁸F nuclear reaction with a proton beam of 19 MeV and enriched [18O]H₂O (30 %) as target material. To the irradiated target water (370 MBq, 10 mCi, 1 ml) is added a mixture of Kryptofixe(10 mg), potassium carbonate (1 mg), and acetonitrile (0.8 ml). The mixture is heated to 100° C. under a stream of nitrogen. After the removal of solvent, acetonitrile (0.5 ml) is added and again evaporated. This step is repeated twice.

The vial containing anhydrous [¹⁸F]KF-kryptate is cooled to room temperature and a solution of 4-trimethylammonium benzaldehyde trifluoromethylsulfonate (1 mg) in anhydrous DMSO (0.2 ml) is added. The mixture is heated at 90° C. for 15 min and cooled to room temperature.

### Conjugation Step

A solution of deprotected aminooxy-insulin (see above, 1-2 equivalents) in ammonium acetate buffer (pH 4.0, 5 mM, 0.1 ml) is added followed by heating at 70° C. for 10 min. The reaction mixture is quenched with HPLC mobile phase (0.2 ml, 20% B) and purified by preparative HPLC.

### Preparation of (BOC-Aminooxy-Acetylamine)-Hexanoic Acid (Compound 3, Scheme 2)

N-succinimidyl BOC-3-(Aminooxy)acetate is obtained from BOC-3-(aminoxy)acetic acid according to the method described by S. Deroo et al. [Tetr. Lett. 44 (2003) 8379]. The N-succinimidyl ester is reacted with 7-aminoheptanoic acid (1.1 equivalents) and diisopropylethyl amine (3 equivalents) in dichloromethane for 16 hours at room temperature. The coupling product, Compound 3, is purified by flash chromatography on silica.

Preparation of B₁-(BOC-Aminooxy-Acetylamine)-Hexanoyl-A₁, B₂₉-di-BOC-Insulin (Compound 4, Scheme 2)

The coupling of the aminooxy linker is carried out as described above for B₁-BOC-aminooxy-A₁,B₂₉-di-BOC-insulin (Intermediate 1).

### Preparation of B₁-(4-Fluoro-Benzylideneaminooxv-Acetylamine)-Hexanoyl-Insulin (Compound 5, Scheme 2)

The removal of the BOC protecting groups of the insulin precursor and the subsequent coupling with 4-fluorobenzaldehyde is carried out as described above for Compound 1.

### Preparation of B₁-(4-[¹⁸F]Fluoro-Benzylideneaminooxy-Acetylamine)-Hexanoyl-Insulin (Compound 6, Scheme 3)

The radiosynthesis of Compound 6 from Compound 4 and [¹⁸F]-4-fluorobenzaldehyde is carried out as described above for the preparation of Compound 2.

What is claimed is:

1. A method for radiolabelling comprising reaction of a compound of formula (I) with a compound of formula (II) 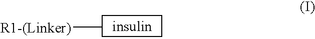
R*−(Linker)−R2 (II)
wherein
R1 is a functional group which reacts site-specifically with R2. R1 can be ammonia derivatives such as primary amine, secondary amine, hydroxylamine, hydrazine, hydrazide, aminoxy, phenylhydrazine, semicarbazide, or thiosemicarbazide, and is preferably a hydrazine, hydrazide or aminoxy group;
R2 is an aldehyde moiety, a ketone moiety, a protected aldehyde such as an acetal, a protected ketone, such as a ketal, or a functionality, such as diol or N-terminal serine residue, which can be rapidly and efficiently oxidised to an aldehyde or ketone using an oxidising agent;
R* is a radiolabel moiety suitable for detection by SPECT or PET;
to give a conjugate of formula (III): 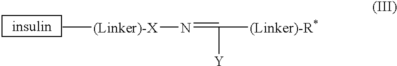
wherein X is —CO—NH—, —NH—, —O—, —NHCONH—, or —NHCSNH—, and is preferably —CO—NH—, —NH— or —O—; Y is H, alkyl or aryl substituents; R* is as defined for the compound of formula (II).

2. A method according to claim 1 wherein R* is ¹⁸F, radioiodine (¹²³I, ¹²⁴I, ¹²⁵I, or ¹³¹I), ⁷⁵Br, or a ¹¹C containing group such as [¹¹C]C₁₋₆ alkylamine.

3. A method according to claim 1 wherein R* is ¹⁸F.

4. A method for radiofluorination comprising reaction of a compound of formula (Ia): 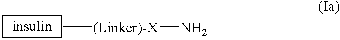
wherein X is —CO—NH—, —NH— or —O— and is preferably —O—; with a compound of formula (IIa): 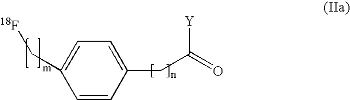
wherein m is an integer of 0 to 10, n is an integer of from 0 to 20, and Y is hydrogen, C₁₋₆alkyl, or phenyl;
to give a compound of formula (IIIa): 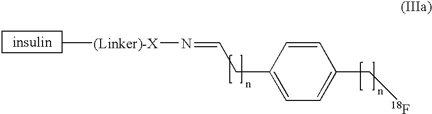
wherein X is as defined for the compound of formula (Ia), m and n are defined as for the compound of formula (IIa).

5. A method according to claim 4 comprising reaction of a compound of formula (Ib): 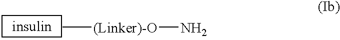
with a compound of formula (IIb): 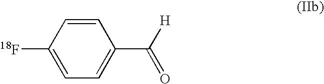
to give a compound of formula (IIIb):

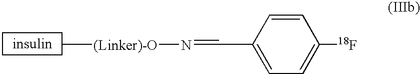

6. A compound of formula (III): 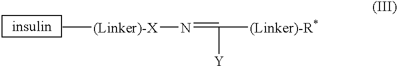
wherein X is —CO—NH—, —NH—, —O—, —NHCONH—, or —NHCSNH—, and is preferably —CO—NH—, —NH— or —O—; Y is H, alkyl or aryl substituents; R* is a radiolabel moiety suitable for detection by SPECT or PET.

7. A compound according to claim 6 wherein R* is ¹⁸F, radioiodine (¹²³I, ¹²⁴I, ¹²⁵I, or ¹³¹I), ⁷⁵Br, or a ¹¹C containing group such as [¹¹C]C₁₋₆alkylamine, and is preferably ¹⁸F.

8. A compound according to claim 6 of formula (IIIa): 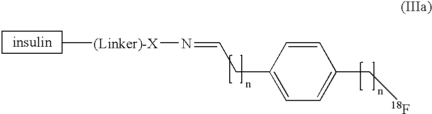
wherein X is —CO—NH—, —NH— or —O—; m is an integer of 0 to 10; n is an integer of from 0 to 20.

9. A compound according to claim 6 of formula (IIIb):

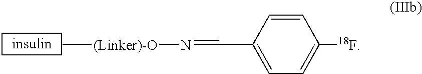

10. A compound of formula (I) 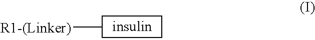
wherein R1 is an ammonia derivative such as primary amine, secondary amine, hydroxylamine, hydrazine, hydrazide, aminoxy, phenylhydrazine, semicarbazide, or thiosemicarbazide, and is preferably a hydrazine, hydrazide or aminoxy group.

11. A compound according to claim 10 of formula (Ia): 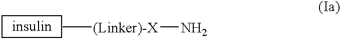
wherein X is —CO—NH—, —NH— or —O—.

12. A compound according to claim 10 of formula (Ib):

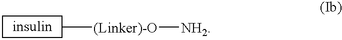

13. A radiopharmaceutical composition comprising an effective amount of a compound of formula (III), (IIIa), or (IIIb) as defined in claim 6, together with one or more pharmaceutically acceptable adjuvants, excipients, or diluents.

14. A compound of general formula (III), (IIIa), or (IIIb) as defined in claim 6, for medical use.

15. The method of use of a radiolabelled conjugate of formula (III), (IIIa), or (IIIb) as defined in claim 6 for the manufacture of a radiopharmaceutical for use in a method of in vivo imaging, suitably SPECT or PET, for imaging a disease in which insulin is implicated

16. A method of generating an image of a human or animal body involving administering a radiopharmaceutical to said body, e.g. into the vascular system and generating an image of at least a part of said body to which said radiopharmaceutical has distributed using SPECT or PET, wherein said radiopharmaceutical comprises a radiolabelled conjugate of formula (III), (IIIa), or (IIIb) as defined in claim 6.

17. A method of monitoring the effect of treatment of a human or animal body with a drug to combat a condition associated with insulin, said method comprising administering to said body a radiolabelled conjugate of formula (III), (IIa), or (IIIb) as defined in claim 6 and detecting the uptake of said conjugate, said administration and detection optionally being effected repeatedly.
